(19) United States
(12) Patent Application Publication
(10) Pub. No.: US 2010/0069438 A1
(43) Pub. Date: Mar. 18, 2010

(54) ANESTHESIA INDUCTION AND MAINTENANCE DRUG

(75) Inventor: Randall S. Hickle, Lubbock, TX (US)

Correspondence Address: HOGAN & HARTSON LLP;IP GROUP, COLUMBIA SQUARE, 555 THIRTEENTH STREET, N.W., WASHINGTON, DC, 20004, US

(73) Assignee: SCOTT LABORATORIES, INC., Lubbock, TX (US)

(21) Appl. No.: 12/522,656

(22) PCT Filed: Sep. 15, 2006
(86) PCT No.: PCT/US06/36189

§ 371 Date: Aug. 12, 2009

Related U.S. Application Data
(60) Provisional application No. 60/722,203, filed on Sep. 30, 2005.
(60) Provisional application No. 60/718,148, filed on Sep. 16, 2005.

(51) Int. Cl. A61K 31/445; A61P 29/00

(52) U.S. Cl. 514/329

(57) ABSTRACT

Disclosed herein is a combination drug designed for use by anesthesiologists for the purposes of inducing and/or maintaining general anesthesia and minimizing the stress response to pain that results from the noxious stimuli associated with surgical procedures. The present invention is suitable for use in general anesthesia to decrease the need for muscle relaxant administering and permit patients to drive themselves home after receiving the anesthetic. This invention includes a combination drug comprised of propofol (2,6-diisopropylphenol) and remifentanil (N-phenyl-N-(4-piperidinyl)amides) in which the preferred embodiment contains 2 mcg of remifentanil for each 1 mg of propofol. The combination drug invention may include a package as two drugs of proper proportions shipped and sold in a two compartment vial or syringe for mixing immediately prior to use. Alternatively, the invention may comprise a single drug prepared with proper preservatives in an aqueous solution.

### CROSS REFERENCE TO RELATED APPLICATIONS

This application claims the benefit of priority of co-owned U.S. Provisional Patent Application Ser. Nos. 60/718,148, filed Sep. 16, 2005, and 60/722,203, filed Sep. 30, 2005.

### FIELD OF THE INVENTION

The present invention is primarily directed to a combination drug for inducing and maintaining general anesthesia.

### BACKGROUND OF THE INVENTION

For years, general anesthesia was normally induced with an intravenous injection of fast acting sedative-hypnotic agents such as water-soluble barbiturates which include, among others, thiopental, methohexital, and thiamylal. More recently, however, the use of these barbiturates has, in many anesthesia practices, been replaced with propofol. The use of the later is believed to result in less postoperative nausea and vomiting and in a faster recovery.

After induction with propofol, general anesthesia is often maintained by halogenated inhalational agents and nitrous oxide that provide both hypnosis and analgesia, and when a greater level of analgesia is required, opioid narcotic agents such as morphine or fentanyl may be used to supplement the inhalational drug. In the alternative, the state of general anesthesia may be maintained by an intravenous infusion of propofol in combination with an opioid narcotic.

For initiation of general anesthesia, the substitution of propofol for barbiturates is believed by many clinicians to be a step forward. However, the use of propofol to induce general anesthesia is not without problems. Propofol is a sedative and has a minimal analgesic effect. It does little to attenuate the body's stress response to pain. Consequently, when anesthesia is induced by propofol, the patient may be unconscious and amnestic, but the body still experiences and undergoes undesirable stress responses to pain. Usually, some degree of pain and associated stress response occurs after general anesthesia is induced with propofol, either at the time of tracheal intubation or upon initiation of the surgical procedure. The stress response to pain is often noted by a rise in the patient's blood pressure and heart rate. In patients with coronary artery disease, this rise in blood pressure and heart rate can increase myocardial oxygen consumption in the presence of limited oxygen supply, thereby resulting in myocardial ischemia and/or infarction. In patients with aneurismal dilation of arteries (e.g., in patients with a cerebral aneurysm) the elevation in blood pressure can cause life-threatening bleeding.

Efforts to avoid such problems include the use of beta blockers. Studies suggesting that the addition of beta blockers may mitigate the stress response of surgery are noted in the Newsletter of the Anesthesia Patient Safety Foundation, volume 17, No. 2, 21-32 dated Summer 2002. While the studies suggest that beta blockers would be helpful, such imposes the costs and requirements of administering a separate, additional drug which has a profile of side effects and potential adverse consequences in and of itself. Moreover, the results from uses of these beta blockers for all patient subgroups are not regarded as conclusive. As reported, “The various patient subgroups which might yield a positive cost-benefit analysis from aggressive preoperative testing remain to be identified.” See Newsletter, supra. Furthermore, it seems oxymoronic to treat the effect of unmitigated pain with a beta blocker that merely blocks the symptoms caused by stress hormones rather than to treat the cause itself with analgesic medicaments that minimize the stress response itself.

General anesthesia during a medical procedure usually imposes the requirement that a patient be driven home after the procedure and such is often a further imposition or hardship upon the patient and his family. Recovery from general anesthesia is dependent upon the dissipation of sedative and analgesic drug effects. Over the course of induction and maintenance of general anesthesia, propofol accumulates in body tissues, causing delays in recovery. Remifentanil, on the other hand, is so rapidly metabolized that it does not significantly accumulate over the course of anesthetic administration. The use of remifentanil and propofol together is synergistic, and thus, less propofol is required to achieve a desired level of drug effect. If a short-acting opioid analgesic such as remifentanil is used to supplement induction and maintenance of general anesthesia with propofol, the patient's recovery is significantly shortened, increasing the likelihood that he can normally drive home alone. Again, however, the administration of two separate drugs is time consuming, tedious and results in further delay.

In most cases in which a patient is to be intubated after general anesthesia has been induced, the anesthesiologist will also administer a muscle relaxant such as succinylcholine or atracurium. Such will relax the muscles to facilitate tracheal intubation and in addition the muscle relaxant may facilitate a quiet surgical field for the surgeon. Muscle relaxants have medical risks and potential adverse consequences. Moreover, such muscle relaxants do not eliminate the stress response to pain, as evidenced in the rise in the patient's blood pressure and heart rate nor eliminate the possibility of myocardial ischemia and/or infarction.

In the past, efforts have been made to avert these problems of stress and resulting increases in heart rate and blood pressure by sequential administration of remifentanil and propofol. Such appears to be an improvement, but the associated calculations of dosages and preparation of the two drugs and the sequential administration is tedious, time consuming, and problematic.

In my prior patent application Ser. No. 09/324,759 filed Jun. 3, 1999 that is based upon a provisional application filed Jun. 3, 1998, the concept of mixtures of several drugs was disclosed for the purpose of computer assisted sedation of a patient during a medical procedure by the procedural physician without the assistance of an anesthesiologist. Those mixtures included propofol and remifentanil and the intended application was that of providing sedation, below the level of general anesthesia, to a non-intubated, spontaneously-ventilating patient during a painful medical procedure. Since remifentanil can result in ventilation suppression of the patient and since this side effect is potentiated by co-administration of propofol, the ratio of remifentanil to propofol had to be low to achieve the intended purposes disclosed in my prior patent application Ser. No. 09/324,759 filed Jun. 3, 1999, in view of the absence of an anesthesiologist skilled in the maintaining ventilation that might result from the suppression of the patient's intrinsic drive to breathe. Such an admixture contains insufficient remifentanil to optimally achieve or maintain a general anesthetic state as intended by this disclosure and invention.

In December of 1999, Joo et. al. filed an application that matured into U.S. Pat. No. 6,071,933 that disclosed the concept of a combination drug comprised of propofol and remifentanil in an amount effective to sedate and control pain while simultaneously maintaining a mammal in a semi-conscious state. Joo et. al. refers to the admixture as REMIPRO and describes it as having the advantage of providing pain relief to the patient without becoming unconscious. According to Joo, et. al., the suggested admixture is designed for use by the patient in conjunction with a patient controlled anesthesia (PCA) modality.

In 1998, the Department of anesthesiology of the Brook Army Medical Center in San Antonio, Tex. is reported to have infused propofol some sixty seconds after the infusion of remifentanil in a group of patients undergoing general anesthesia and intubation, but, as noted above, such required the inconvenient calculations and preparation of the two separate drugs and a time delay in administration of the second drug (see Anesthesia Analg, January 1998 (1), 45-9).

### SUMMARY OF THE INVENTION

To overcome these and other problems, the present invention of a combination drug of propofol and remifentanil facilitates the administration of the two drugs and also resolves the problems associated with patient stress caused by infusion of propofol alone. Thus, the primary intended benefit of this invention is to provide a combination drug for inducing and maintaining general anesthesia, facilitating intubation without muscle relaxants and achieving a quiet surgical field—while eliminating or substantially reducing stress and the resulting the dangers of increased heart rate and blood pressure and myocardial ischemia and/or infarction and hemorrhagic strokes. Another benefit of this combination drug is to achieve a fast offset of general anesthesia so as to enable the patient to be timely discharged and potentially to drive himself home. A further benefit is to simplify the administration of the anesthetic by the use of a single drug, simple calculations of the dosage, a single injection and a resulting fast onset of general anesthesia.

The present invention is a combination drug designed for use by anesthesiologists for the purposes of inducing and/or maintaining general anesthesia and minimizing the stress response to pain that results from the noxious stimuli associated with surgical procedures, because this stress response may cause myocardial ischemia and/or infarction and hemorrhagic strokes. It is also designed for use in general anesthesia that decreases the need for muscle relaxant use, and it is designed for use as a drive-home anesthetic. This invention includes a combination drug comprised of propofol (2,6-diisopropylphenol) and remifentanil (N-phenyl-N-(4-piperidinyl) amides) in which the preferred embodiment contains 2 mcg of remifentanil for each 1 mg of propofol. For convenience and to facilitate use, this combination drug invention may include a package as two drugs of proper proportions shipped and sold in a two compartment vial or syringe for mixing immediately prior to use. Alternatively, the invention may comprise a single drug prepared with proper preservatives in an aqueous solution.

Accordingly, the goals and objectives of this invention are, among other things:

To provide a method of inducing general anesthesia and intubating a patient while minimizing the patient's stress response to pain;

To provide a new combination drug for inducing general anesthesia that will minimize a patient's stress response to painful stimuli, including among others, tracheal intubation and surgical incision and provide a quiet surgical field;

To provide a new combination drug for efficiently inducing and maintaining general anesthesia that can be packaged in a single vial or syringe and administered by an anesthesiologist with the use of a single standard syringe or an infusion pump.

To provide a new combination drug for efficiently inducing and maintaining general anesthesia that provides fast onset of the state of general anesthesia as well as fast recovery of full consciousness;

To provide a pre-packaged combination of two properly proportioned drugs in a single, two compartment syringe or vial to facilitate mixing and infusion into a patient, or alternatively, to provide a combination drug in an aqueous solution for efficient induction and/or maintenance of general anesthesia of a patient; and

To provide a combination drug in a single vial that can be used in conjunction with a programmable infusion pump to induce and to maintain a patient in the state of general anesthesia.

### BRIEF DESCRIPTION OF THE DRAWINGS

FIG. 1 is a side elevational view, drawn through a vertical plane, of a two compartment vial depicting a preferred embodiment of the packaged combination drug product of this invention; and

Figure two is a side elevational view, drawn through a vertical plane, of an alternative embodiment of the invention.

### DETAILED DESCRIPTION OF THE PREFERRED EMBODIMENTS

The manner in which the foregoing objectives are achieved is set forth in this detail description so as to fully enable those skilled in the art to make and use the proposed combination drug for the purposes intended.

The present invention is a combination of two existing drugs, propofol and remifentanil. Propofol is chemically described as 2,6-diisopropylphenol and is often sold as an emulsion by Astra-Zeneca under the trademark Diprivan®. It is a sedative-hypnotic agent with a short context sensitive half time that is effective to induce and maintain general anesthesia, but it has minimal analgesic properties. Although a current, primary application of propofol is the induction of general anesthesia, this agent does not sufficiently attenuate the body's response to the pain of intubation or the surgical procedure thus permitting an increase in blood pressure and heart rate along with other consequences of inadequate analgesia. Lacking an analgesic effect, propofol inductions require the addition of muscle relaxants to facilitate tracheal intubation as the patient will move and cough if intubated after induction with propofol alone. On the other hand, remifentanil (N-phenyl-N-(4-piperidinyl)amides) is an analgesic opioid rather than a sedative-hypnotic agent, and it has the capability of precluding the patient's reaction to pain. Significantly, remifentanil has a very rapid onset and a rapid offset regardless of duration of administration. Indeed, both drugs are fast acting, and both have a short half life that permits the patient to quickly regain full consciousness.

When used together, the combination has further unexpected benefits. The combination is synergistic in that less propofol is needed to place the patient under general anesthesia and to maintain this level of effect, resulting in less propofol accumulation in the body over the course of an anesthetic. Less remifentanil is needed to eliminate the pain associated with intubation and surgical pain. Consequently, this drug combination provides a low cost method of eliminating pain and maintaining unconsciousness in the process of achieving general anesthesia.

A preferred and effective combination drug is comprised of a ratio of two micrograms of remifentanil to each milligram of propofol. Such can be injected into the vein with a syringe or an infusion pump to effectuate general anesthesia to permit intubation with minimal risk of unacceptable increases in blood pressure or heart rate that can cause myocardial ischemia and/or infarction or hemorrhagic stroke. This combination drug also permits intubation without the requirement of muscle relaxants, and, as previously stated, it has the potential to permit the patient to drive himself home in many cases.

An example of the effectiveness of the combination drug is demonstrated by the following example:
50 ml of propofol (10 mg/ml) was drawn into a 60 ml syringe. 3 ml of this propofol was repeatedly injected into a 1 mg vial of remifentanil powder to reconstitute the drug in propofol, and this reconstituted fluid was redrawn into the 60 ml syringe. Thus, a mixture was created containing 50 ml of fluid with 500 mg of propofol and 1 mg of remifentanil in concentrations of 10 mg/ml propofol and 20 mcg/ml of remifentanil. General anesthesia was induced when 20 ml of this combination mixture was injected into a 100 kg patient (200 mg propofol and 400 mcg remifentanil). He went to sleep within 30 seconds and was intubated easily and uneventfully at 90 seconds in the absence of muscle relaxants. His blood pressure dropped from 130/85 to 95/75 and his heart rate fell from 85 to 60. Subsequently, general anesthesia was maintained with a continuous infusion of this same combination mixture at a rate of 0.75 ml/minute. Positive pressure ventilation was required, because the patient exhibited no spontaneous ventilatory effort at an end tidal CO₂ of 48. Surgery commenced without autonomic signs of stress (the heart rate and blood pressure did not increase), and the patient did not move. At the completion of the surgery, the infusion of this mixture was discontinued, and the patient regained spontaneous ventilation at an end tidal CO2 of 43 and the patient awakened and was extubated 8 minutes after cessation of the infusion of this mixture of drugs. The patient had a rapid and clear-headed emergence from general anesthesia and was discharged to home within two hours.

As noted above, one effective ratio of remifentanil to propofol is 2 mcg of remifentanil to 1 mg of propofol. As expected in most cases, an effective dosage will be dependent upon the age and medical condition of the patient, adjuvant drug levels, and the level of stimulation to be produced by the intended procedure.

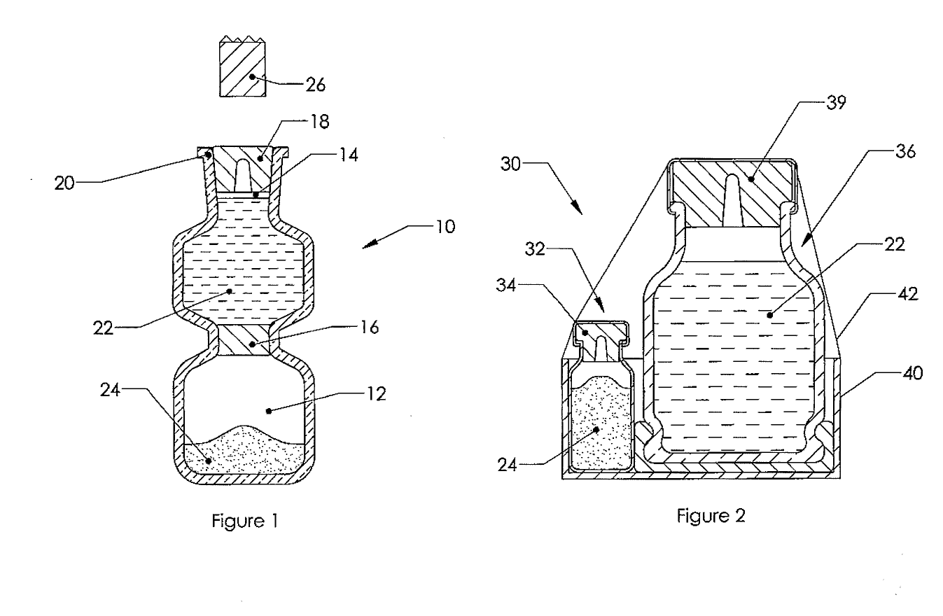

One efficient method of providing the combination drug to the anesthesiologist is to utilize a package comprising a two compartment vial such as that disclosed in FIG. 1. Such two compartment vials are well known in the art and are illustrated by U.S. Pat. Nos. 5,405,001 and 4,331,233, each of which is incorporated by reference herein. In one embodiment of this invention, 400 mcg of lyophilized remifentanil power (24) is placed in the lower compartment (12) of the two compartment container (10) and 20 cc of a lipid emulsion containing 10 mg/cc of propofol (22) are placed in the upper compartment (14), the two compartments and drugs being separated by a stopper (16) in conventional fashion until use by the anesthesiologist.

At the time of use, a piston plunger (26) is pressed downward pressurizing the lipid emulsion which then imposes a force on the intermediate stopper (16) to dislodge it and permit the emulsion (22) to mix with the lyophilized remifentanil powder (24). At that time, the immiscible liquid emulsion containing the emulsion droplets of propofol (22) will dissolve the lyophilized remifentanil powder (24) and the resulting mixture will contain a ratio of 2 mcg of Remifentanil to 1 mg of propofol and the resulting mixture will contain 20 mcg/cc of remifentanil and 10 mg/cc of propofol.

Subsequent to dissolving the powder, the resulting mixture can be drawn into a syringe and utilized to induce general anesthesia. Thus, a single bolus injection of the combination drug simplifies the induction of general anesthesia where, but for this invention, a first injection of propofol would be utilized and a second injection of a muscle relaxant would be used. And in spite of the two injections, due to inadequate analgesia the patient can be expected to incur stress, higher blood pressures and heart rates. For some patients, such would further result in myocardial ischemia and/or infarction.

An alternative, beneficial use of this combination drug would be to sell it in a larger, two compartment vial ready for mixing of a lyophilized power of remifentanil with a propofol emulsion. After mixing, the combination drug could be infused into the patient with a conventional programmable infusion pump that is programmed to first administer a bolus to induce general anesthesia and then to maintain general anesthesia throughout the medical procedure.

Another alternative embodiment is disclosed in connection with FIG. 2. This embodiment comprises a package (30) of two conventional vials (32) and (36). The smaller vial (32) preferably contains 1 mg of remifentanil powder (24) and the larger vial (36) contains 500 mg of propofol in an emulsion (22) of 50 ml. For convenience, the two drugs are packaged together in a cardboard container (40) with a shrink wrapped film (42) providing package integrity for shipment, storage and shelf life. For convenience, simplified use and infusion, a syringe (not shown) can be used to draw the emulsion (22) through the plug (39) and inject a portion, say 3 ml, into the smaller vial (32) for dissolving the remifentanil powder (24) and mixing the two drugs. The resulting mixture is then drawn from the smaller vial (32) into the syringe. Thus, a mixture is easily created in the syringe containing 50 ml of fluid with 500 mg of propofol and 1 mg of remifentanil in concentrations of 10 mg/ml propofol and 20 mcg/ml of remifentanil—a mixture having the preferred ratio for inducing and maintaining general anesthesia. As those skilled in the art will appreciate, the two drugs can also be sold separately in a two compartment syringe, or in a vial and syringe package.

Upon completion of the medical procedure and termination of the infusion, the patient will quickly regain full consciousness with a minimum probability of nausea. This cocktail of propofol plus remifentanil, requiring decreased dosing of both agents due to the profound synergistic effect of the combination, comprises part of a technique that can accomplish a drive-home general anesthetic. When appropriate, an additional component of this drive-home technique is the use of local anesthetic agents for postoperative pain relief. An additional component of this technique is post-procedure testing of the patient's cognitive and motor function to ensure adequate performance prior to discharge. Pre-procedure testing to establish baseline performance metrics can be combined with post-procedure testing to increase the sensitivity and specificity of such evaluations. Cognitive and motor function can be tested in manual or automated fashion, and the approach is very similar to the assessment performed by a police officer when evaluating a person suspected of driving under the influence of alcohol.

As will be appreciated by those skilled in the art, the two drugs can also be mixed together in formulation with other preservative compounds to provide adequate shelf life as a mixture. A stable co-formulation of propofol and remifentanil may be achieved by one of several methods. For example, use of organic co-solvents, ethanol, glycerol, and propylene glycol alone or in combination in mixtures with water (water for injection, USP) may be used to co-formulate propofol and remifentanil hydrochloride. Ethanol may be used at concentrations up to 20%, glycerol may be used in concentrations up to 50%, and propylene glycol may be used at concentrations up to 30% in the final dosage form. The final dosage form may contain ratios up to 50% organic ingredients. The propofol would be dissolved in the required amount of the organic solvent, the remifentanil hydrochloride would be dissolved in the required amount of water. The two solutions would then be combined to produce the final formulation.

Other organic carriers may be used to solubilize the propofol in aqueous media including small organic molecules such as sugars (sucrose, dextrose, lactose, trehalose, and mannitol) or polymers such as povidone (polyvinylpyrrolidone) or polyethylene glycol (PEG). Sugars may be used in concentrations up to about 5% w/w of the final formulation (to be isotonic for example). The povidone may be used in concentrations of 10 to 25% w/w of the final formulation. The polyethylene glycol (PEG 300 or PEG 400) may be used in concentrations up to 30% v/v of the final formulation. To co-formulate propofol and remifentanil, the carrier would be dissolved in water followed by the remifentanil and then the propofol to produce the final formulation.

Another category of carriers are those that form host-guest complexes. These include alfa and beta-cyclodextrin, sulfobutyl-ether beta cyclodextrin, and hydroxypropyl beta cyclodextrin. The cyclodextrins are typically used at concentrations of 10 to 30% w/w of the final formulation. The remifentanil would be dissolved in water. The propofol would be blended with the cyclodextrin. The aqueous solution would then be added slowly and with vigorous mixing to the propofol-cyclodextrin blend and mixing continued until a solution was achieved. Various agents, typically used in pharmaceutical preparations, could then be added to modify the pH of the formulation.

Propofol can be formulated as an oil-in-water emulsion using oil (soy bean or sesame oil), propylene glycol, and a lecithin (egg or soy lecithin) and water. Remifentanil can be formulated as a lyophilized preparation using caking agents such as mannitol or dextrose. Agents to modify the pH of the final formulation can also be added including citric acid or sodium citrate, acetic acid or sodium acetate, sodium hydroxide, or hydrochloric acid. The propofol can be packaged in a pre-filled syringe and the remifentanil in a single-dose vial. The propofol emulsion can then be used to re-constitute the vial of remifentanil at the time of use to provide the final formulation.

Alternatively, the free-base form of remifentanil may be prepared and incorporated into the oil-phase of the propofol oil-in-water emulsion. For example, remifentanil may be dissolved in water and treated with ammonium hydroxide which will induce the precipitation of the remifentanil free-base. The remifentanil free-base may be isolated by filtration and dried. The propofol and remifentanil may then be dissolved in the oil and glycerol and mixed until dissolved. The lecithin is suspended in water and blended. The oil solution is then added to the water lecithin suspension and homogenized until the proper particle size distribution is achieved (typically below 1 μm). Various agents, typically used in pharmaceutical preparations, could then be added to modify the pH of the solution.

As noted above, persons skilled in the art will appreciate that the package can take numerous and various forms that extend from a single mixture co-formulation to a variety of two compartment vials and syringes for mixing just prior to use. All will facilitate administration of the drug and eliminate stress resulting from intubation and permit quick recovery from general anesthesia.

1. A product for efficiently facilitating the induction of general anesthesia and intubation, said product comprising:
a) a package containing a combination of drugs;
b) said combination of drugs in said package comprising remifentanil and propofol in a ratio effective to induce general anesthesia so as to permit intubation of said patient without stress, increased heart rate and/or increased blood pressure.

2. A product as recited in claim 1 in which said package comprises two compartments container in which
a) one compartment contains a lyophilic power of remifentanil;
b) the other compartment contains an emulsion of propofol; and
c) said compartments are sealed one from another in a manner that permits them to easily be mixed at or near the time of administration.

3. A product as recited in claim 1 in which said combination of drugs are combined in an aqueous solution.

4. A product as recited in claim 1 in which said package comprises two vials, one of which contains a lyophilic powder of remifentanil and the other contains an emulsion of propofol in a ratio of about 2 mcgs of remifentanil to 1 mg of propofol.

5. A product as recited in claim 1 in which said package comprises in which said two compartments comprise a single syringe having a single compartment and a vial having a single compartment.

6. A method of inducing general anesthesia in a patient comprising the steps of:
a) drawing a combination drug mixture of remifentanil and propofol; and
b) infusing said drug into said patient to induce general anesthesia without pain.

7. A method as recited in claim 6 in which said mixture comprises a ratio of about 2 mcg of remifentanil to 1 mg of propofol.
